(19) United States
(12) Patent Application Publication
(10) Pub. No.: US 2004/0115209 A1
(43) Pub. Date: Jun. 17, 2004

(54) Compound targeted for specific cells with reduced systemic toxicity

(76) Inventors: Uri H. Saragovi, Montreal (CA); Veronique Guillemard, Montreal (CA)

Correspondence Address: GREENBERG TRAURIG, LLP, ONE INTERNATIONAL PLACE, 20th FL, ATTN: PATENT ADMINISTRATOR, BOSTON, MA, 02110, US

(21) Appl. No.: 10/600,623

(22) Filed: Jun. 20, 2003

Related U.S. Application Data
(63) Continuation of application No. PCT/CA01/01854, filed on Dec. 21, 2001.
(60) Provisional application No. 60/256,987, filed on Dec. 21, 2000.

(51) Int. Cl. A61K 48/00; A61K 39/395; A61K 31/525

(52) U.S. Cl. 424/178.1; 514/34; 514/44; 514/251; 530/391.1

(57) ABSTRACT

The present invention relates to a compound to selectively kill or protect a target cell in a patient with reduced systemic toxicity, which comprises a compound of the formula: W-Z-X wherein, X is a toxic agent or protective agent; W is a biologically active molecule which is adapted to selectively bind the target cell directly or indirectly; and Z is a breakable linker which covalently links W and X together, wherein the linked W remains bioavailable and bioactive, whereby the breakable linker releases the toxic agent or protective agent into the cell.

### BACKGROUND OF THE INVENTION

(a) Field of the Invention

The invention relates to a compound as a therapeutical agent to target and kill or protect specific cells in a patient with reduced systemic toxicity.

(b) Description of Prior Art

The clinical use of chemotherapeutic agents against malignant tumors is successful in many cases, but also has several limitations. These agents do not affect tumor cell growth selectively over rapidly growing normal cells, leading to high toxicity and side effects. For example, paclitaxel and related taxanes are a very potent class of anticancer drugs first isolated in 1971. Paclitaxel has a unique mechanism of action, it promotes microtubule polymerization leading to abnormaly stable and nonfunctional microtubules. Hence, cells are blocked at the G2-M phase of the cell cycle, leading to apoptotic death.

Paclitaxel has clinical efficacy, despite several problems associated with poor solubility and high toxicity. Clinical trials showed remarkable efficacy against advanced solid tumors such as ovarian and breast cancer and a panel of other tumors. Most of the side effects of taxanes occur at rapidly growing tissue such as bone marrow, hematopoietic, and gut epithelia. Because microtubule function is key for neuronal survival, neurotoxicity is also a problem for taxanes.

Doxorubicin is one of the most widely used anticancer agent. It has a strong antiproliferative effect over a large panel of solid tumors. Doxorubicin intercalates into DNA and breaks the strands of double helix by inhibiting topoisomerase II.

Despite its clinical efficacy, Doxorubicin suffers a major drawback which is common of all chemotherapeutic agents: it is not tumor selective and therefore affects healthy tissue as well causing severe side effects, including cardiotoxicity and myelosuppression (Tewek K. M. et al. Science 226: 466468, 1984). Moreover, the intrinsic or acquired resistance of cancer cells to Doxorubicin is another factor that limits its efficacy. For instance, the multidrug resistance associated p-glycoprotein (p-gp) is a transmembrane pump that facilitates active cellular efflux of toxic compounds and, thereby, lowers cytotoxicity of the drugs (Zhang D. W. et al. J. Biol. Chem. 276(16): 13231-9, 2001). Verapamil is a p-glycoprotein inhibitor commonly used in drug resistance studies.

Several approaches have been developed in order to specifically deliver Doxorubicin to tumor cells using monoclonal antibodies (mAbs) or small peptides as carrier molecules. Site-directed delivery may increase the intratumor concentration of the drug while decreasing systemic toxicity.

The outcome of targeted chemotherapy greatly depends on 2 factors: the ability of the carrier molecule to selectively recognize tumor cells and the nature of the chemical linkage used for coupling the cytotoxic agent to the carrier. Ideally, the conjugate should be stable and inactive in the circulation, with the cytotoxic radical released in its active form in the target tumor tissue after internalization of the conjugate (Guillemard V. et al. Cancer Res. 61:694-699, 2001).

Different chemical strategies have been used to couple drugs to mAbs including periodate oxidation of mAbs. Using this approach, diols located in the antibody's carbohydrate chains are cleaved by periodate to form reactive aldehyde groups which can further react with amine or hydrazide residues forming a C—N linkage. The main advantage of this technique is that the carbohydrate residues that are affected are usually located at distant sites from the antibody's binding regions. Thus, modification of the antibody through these residues should have little or no effect on the antibody's activity.

Doxorubicin has previously been coupled to mAbs using this method. The C—N linkage has been shown to be relatively stable in the circulation while Doxorubicin was released inside of the tumor cells presumably following hydrolysis of the conjugate after exposure to low pH and lysosomal/endosomal glycosidases.

The problem of selectivity can be addressed by using monoclonal antibodies (mAbs) that target “tumor markers”, which are proteins generally overexpressed on the surface of tumor cells. In passive immunotherapy, mAbs can act either as pharmacological agents, as adjuvants or as cytotoxic agents upon fixation of complement, and as carriers for large toxins or cytokines. However, mAbs are generally poor pharmaceuticals and are poor cytotoxic agents.

Many cancer cells types overexpress certain cell surface components such as proteins or glycolipids, which are known as tumor markers. Examples include receptor tyrosine kinases such as type I insulin-like growth factor receptor (IGF-IR) and Her2/neu, TrkA, etc. It would be desirable to target chemotherapeutics to these tumor markers to achieve more selective tumor death with lower toxicity.

Another method for reducing toxicity would be to selectively deliver protective agents to non-tumor cells. For example the neurotoxicity caused by the chemotherapeutic agent taxol may be ameliorated by selective delivery of neuroprotective agents to neurons in a manner that does not alter the desired tumor killing of non-neuronal cells.

International application published under No. WO 00/33888 discloses a modified form of a therapeutic agent which comprises a therapeutic agent, an oligdpeptide, a stabilizing group and, optionally, a linker group.

U.S. Pat. Nos. 4,997,913 and 5,084,560 disclose a pH-sensitive immunoconjugates which dissociate in low-pH tumor tissue, comprising a chemotherapeutic agent and an antibody reactive with a tumor-associated antigen.

International application published under No. WO 96/09071A1 discloses a conjugate consisting of an active substance and a native protein which is not considered exogenous. The conjugate is distinguished in that, between the active substance and the protein, there is a linker which can be cleaved in a cell.

U.S. Pat. No. 6,030,997 discloses a pharmaceutically acceptable prodrug which is a covalent conjugate of a pharmacologically active compound and a blocking group, characterized by the presence of a covalent bond which is cleaved at pH values below 7.0.

U.S. Pat. No. 6,140,100 discloses a conjugate of a cell targetting molecule and a mutant human caboxypeptidase A enzyme.

U.S. Pat. No. 5,208,323 discloses an antitumor compound which comprises an antibody used to target the anti-tumor agent to the malignant cells.

It would be highly desirable to be provided with a compound as a therapeutical agent to target and kill or protect specific cells in a patient with reduced systemic toxicity.

Furthermore, it would be highly desirable to be provided with the chemical covalent linking of mAbs and chemotherapeutics or mAbs and protective agents, for example, to allow for the delivery of a cytotoxic agent to tumor cells with reduced systemic toxicity.

Furthermore, it would be desirable to be provided with any other ligand conjugated to chemotherapeutics or protective agents in a similar manner.

### SUMMARY OF THE INVENTION

One aim of the present invention is to provide a compound as a therapeutical agent to target and kill specific cells in a patient with reduced systemic toxicity.

Another aim of the present invention is to provide the chemical covalent linking of mAbs and chemotherapeutics or mAbs and protective agents, for example, to allow for the delivery of a cytotoxic agent to tumor cells with reduced systemic toxicity or delivery of protective agents to non-tumor cells.

In accordance with the present invention there is provided a compound to selectively kill or protect a target cell in a patient with reduced systemic toxicity, which comprises a compound of the formula:

W-Z-X

wherein,

X is a therapeutical agent selected from the group consisting of chemotherapeutic agent, antiviral agent, antibacterial agent, antifungal agent and enzyme inhibitor agent;

W is a molecule which is adapted to selectively bind the target cell directly or indirectly; and

Z is a breakable linker which covalently links W and X together, wherein the linked W remains available for binding to the target cell, whereby the breakable linker releases the therapeutical agent into the target cell.

The compound in accordance with a preferred embodiment of the present invention, wherein the compound when bound to the target cell is internalized into the target cell.

The compound in accordance with a preferred embodiment of the present invention, wherein the linker is breakable by pH modification, reduction or enzymatic hydrolysis.

The compound in accordance with a preferred embodiment of the present invention, wherein the chemotherapeutic agent is selected from the group of taxanes, taxanes derivatives, anthracyclines, anthracyclines derivatives, doxorubicin, daunomycin, daunorubicin, adriamycin, methotrexate, mitomycin, epirubicin, nucleoside analogs, DNA damaging agents and tyrphostins.

The compound in accordance with a preferred embodiment of the present invention, wherein the protective agent is an enzyme inhibitor such as a caspase inhibitor agent.

The compound in accordance with a preferred embodiment of the present invention, wherein the therapeutical agent is antisense oligonucleotide or a cDNA for a gene.

The compound in accordance with a preferred embodiment of the present invention, wherein the taxane is paclitaxel.

The compound in accordance with a preferred embodiment of the present invention, wherein the chemotherapeutic agent is doxorubicin.

The compound in accordance with a preferred embodiment of the present invention, wherein the molecule is selected from the group of antibody and mimicking molecules thereof, peptides, peptidomimetics, growth factors, hormones, adhesion molecules, viral proteins and functional fragments thereof.

The compound in accordance with a preferred embodiment of the present invention, wherein the antibody is a monoclonal antibody.

The compound in accordance with a preferred embodiment of the present invention, wherein the antibody binds to a specific receptor on the target cell.

The compound in accordance with a preferred embodiment of the present invention wherein the monoclonal antibody is MC192 (p75 binding), or 5C3 (TrkA binding), or a-IR3 (IGF-1 R binding).

The compound in accordance with a preferred embodiment of the present invention, wherein the compound further comprises a spacer between W and Z and/or between Z and X.

The compound in accordance with a preferred embodiment of the present invention, wherein when W is a primary biologically active molecule indirectly binding to the target cell, the compound further comprises W′ which is a secondary biologically active molecule selectively bound to W and adapted to selectively bind the target cell.

The compound in accordance with a preferred embodiment of the present invention, wherein the primary and/or the secondary biologically active molecules is an antibody.

The compound in accordance with a preferred embodiment of the present invention, wherein a primary antibody is of a species and a secondary antibody is of a different species.

The compound in accordance with a preferred embodiment of the present invention, wherein the secondary biologically active molecule is a rabbit-antimouse antibody.

The compound in accordance with a preferred embodiment of the present invention, wherein the compound is of the formula:

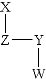

wherein Y is a spacer selected from the group of alkene, alkyl methyl, ethyl ester, ethyl glycol, and, H(CH2CH2O)nOH, n being between 1 and 90.

The compound in accordance with a preferred embodiment of the present invention, wherein the spacer is (CH₂)₃.

The compound in accordance with a preferred embodiment of the present invention, wherein the compound is of the formula I,

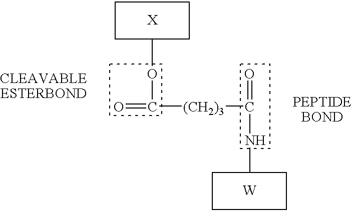

The compound in accordance with a preferred embodiment of the present invention, wherein Z is

The compound in accordance with a preferred embodiment of

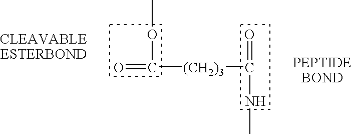

the present invention, wherein the compound is of the formula II,

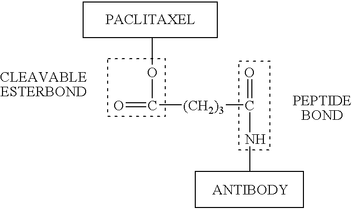

The compound in accordance with a preferred embodiment of the present invention, wherein the compound is of the formula III,

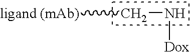

In accordance with the present invention, there is provided a therapeutical composition, which comprises a therapeutically effective amount of a compound of the present invention in association with a pharmaceutically acceptable carrier.

In accordance with the present invention, there is provided an anti-cancer composition, which comprises a therapeutically effective amount of a compound of the present invention in association with a pharmaceutically acceptable carrier, wherein the therapeutical agent is a chemotherapeutic agent.

In accordance with the present invention, there is provided a method for treating cancer with reduced effects in a patient, the method consisting in administering a therapeutically effective amount of a compound of the present invention to a patient, wherein the therapeutical agent is a chemotherapeutic agent.

In accordance with the present invention, there is provided the Use of the compound of the present invention for the manufacture of a medicament for the treatment of cancer with reduced effects in a patient, wherein the therapeutical agent is a chemotherapeutic agent.

In accordance with the present invention, there is provided a method for decreasing toxic side effects and increasing selectivity of a chemotherapeutic agent for tumor cells, the method comprising the step of administering to a patient a conjugate comprising a chemotherapeutic agent conjugated to a molecule which is adapted to selectively bind the target cell directly or indirectly, wherein the compound when bound to the target cell is internalized into the cell and to a breakable linker which covalently links the molecule and the chemotherapeutic agent together, wherein the linked molecule remains available for binding the target cell, whereby the breakable linker releases the chemotherapeutic agent into the target cell.

In accordance with the present invention, there is provided the use of a chemotherapeutic agent conjugated to a molecule for decreasing toxic side effects and increasing selectivity of a chemotherapeutic agent for tumor cells, the molecule being adapted to selectively bind the target cell directly or indirectly, wherein the compound when bound to the target cell is internalized into the cell and to a breakable linker which covalently links the molecule and the chemotherapeutic agent together, wherein the linked molecule remains available for binding the target cell, whereby the breakable linker releases the chemotherapeutic agent into the target cell.

In accordance with the present invention, there is provided a method for by-passing resistance of tumor cells by p-glycoprotein pump (PGP), the method comprising the step of administering the compound of the present invention to a patient in need of such a treatment whereby the biologically active molecule is a monoclonal antibody and the compound avoids membrane diffusion/permeability route to enter into the cells.

In accordance with the present invention, there is provided a compound to selectively protect a target cell which comprises a compound of the formula:

W-Z-X

wherein,

X is a protective agent to cells selected form the group consisting of: enzyme inhibitors such as caspase inhibitors, ligands of nuclear receptors, vitamin D, vitamin E and their analogs, estrogen and its analogs and inhibitors of the apoptotic cascade.;

W is a biologically active molecule which is adapted to selectively bind the target cell directly or indirectly; and

Z is a linker which covalently links W and X together, wherein the linked W remains available for binding the target cell, whereby the linker releases the therapeutical agent into the cell and whereby the compound is providing a patient with a reduced systemic toxicity.

In accordance with the present invention, there is provided a method for decreasing toxic side effects of non-tumor cells, the method comprising the step of administering to a patient a conjugate comprising a protective agent conjugated to a molecule which is adapted to selectively bind the non-tumor target cell directly or indirectly, wherein the compound when bound to the target cell and to a breakable linker which covalently links the molecule and the protective agent together, wherein the linked molecule remains available for binding the target cell, whereby the breakable linker releases the protective agent into the cell and whereby the protective agent internalized in the cell is protecting the cell from subsequent toxicity by a chemotherapeutic agent which is therefore decreasing toxic side effects.

The present invention describe the design, synthesis and evaluation of a targeted cytotoxic conjugate containing Doxorubicin as an agent to kill cells expressing IGF-IR. The mAb α-IR3, selective for IGR-IR, retained full binding and specificity after coupling, and the conjugate delivered Doxorubicin in its active form. The conjugate was more active in vitro and in vivo than free Doxorubicin or free Doxorubicin in combination with mAb α-IR3. Furthermore, the conjugate was highly selective and specific towards cells expressing IGF-IR. Moreover, the conjugate allowed bypassing of the p-glycoprotein-mediated resistance both in vitro and in vivo.

The present invention describes the design, synthesis and evaluation of a targeted cytotoxic conjugate containing Taxol as an agent to kill cells expressing p75 receptors. The mAb MC192, selective for p75, retained full binding and specificity after coupling, and the conjugate delivered taxol in its active form. The conjugate was more active in vitro and in vivo than free taxol or free taxol in combination with mAb p75. Furthermore, the conjugate was highly selective and specific towards cells expressing p75.

The present invention describes the design, synthesis and evaluation of a targeted neuroprotective conjugate containing a caspase inhibitor as an agent to protect neuronal cells expressing TrkA. The mAb 5C3, selective for TrkA, retained full binding and specificity after coupling, and the conjugate delivered the caspase inhibitor in its active form. The conjugate was more active in vitro than free caspase inhibitor. Furthermore, the conjugate was highly selective and specific towards cells expressing TrkA.

### BRIEF DESCRIPTION OF THE DRAWINGS

FIG. 1 illustrates the structure of paclitaxel-antibody conjugate;

FIG. 2 illustrates comparison of dose effectiveness of paclitaxel in continuous presence versus single exposure to drug;

FIG. 3 illustrates cytotoxicity and specificity of paclitaxel-rabbit-antimouse conjugates 4-3.6 (A) and B104 (B);

FIG. 4 illustrates the comparable dose-dependent cytotoxicity of free paclitaxel and paclitaxel-MC192 conjugate;

FIG. 5 illustrates that paclitaxel-MC192 is target selective and specific toward p75-positive cells (A) NIH-3T3 cells were cultured with the indicated molar or molar-equivalent concentrations of drugs and (B) B104 cells were cultured with free paclitaxel (20 nM) or paclitaxel-MC192 (10 nM paclitaxel-equivalent) in the presence or absence of 40 nM unconjugated MC192 mAb;

FIG. 6 illustrates paclitaxel-MC192 conjugates and free paclitaxel arrest cells at the G2-M phase of the cell cycle. B104 cells were untreated or treated with 40 nM MC192, paclitaxel-MC192 conjugates (40 nM paclitaxel-equivalent; 40 nM MC192), or 80 nM free paclitaxel;

FIG. 7 illustrates that paclitaxel-MC192 is efficient at reducing tumor growth and at increasing survival in vivo. Tumor sizes of mice that received five treatments every 2 days of saline (A-C); 130 ng of free paclitaxel (A), 130 ng of free paclitaxel+free MC192 (10 μg; B) or paclitaxel-MC192 (65 ng paclitaxel-equivalent and 10 μg MC192-equivalent; C);

FIG. 8 illustrates the structure of the Doxorubicin-mAb conjugate;

FIG. 9 illustrates comparable dose-dependent cytotoxicity of free Doxorubicin and Doxorubicin-mAb conjugate in vitro;

FIGS. 10A and 10B illustrates the specific toxicity of Doxorubicin-mAb conjugate; and

FIG. 11 illustrates the efficiency of Doxorubicin-mAb conjugate at reducing tumor growth and at increasing survival in vivo.

### DETAILED DESCRIPTION OF THE INVENTION

In accordance with the present invention, there is provided a compound as a therapeutical targeted agent to kill or protect specific cells in a patient with reduced systemic toxicity. Typically lower doses of conjugates may be needed than if the chemotherapeutic agent were to be used alone.

In accordance with a preferred embodiment of the present invention, there is provided the chemical covalent linking of mAbs and chemotherapeutics allowing the delivery of a cytotoxic agent to tumor cells in a patient with reduced systemic toxicity.

In a first embodiment of the present invention, we aimed to fulfill the following five objectives: (a) to chemically conjugate tumor cell specific binding molecules including specific mAbs to the relatively small chemotherapeutic agent; (b) to afford conjugates that are highly soluble in physiological buffers; (c) chemical coupling should not affect mAb targeting function, but should result in inactivation of chemotherapeutic agent activity; (d) after binding to the target receptor mAbs should induce capping and internalization and therefore deliver the conjugate into the tumor cell; (e) the chemical coupling has to allow the release of the chemotherapeutic in its active form after the antibody•cytotoxic drug conjugate is internalized. This is achieved by coupling the chemotherapeutic via a breakable bond which is cleaved after conjugate internalization and exposure to lysozomal vessicles.

We report the synthesis of conjugates paclitaxel•MC192 as an agent to target and kill cells expressing p75 receptors (Guillemard V. et al. Cancer Res. 61:694-699, 2001). We also report the synthesis of paclitaxel-rabbit anti-mouse antibody as an all-purpose secondary reagent that allows selective tumor targeting with the use of any mouse primary antibody. The paclitaxel-coupled antibodies retain high affinity and specificity after conjugation, and the conjugates delivered the cytotoxic agent in its active form. Paclitaxel-Ab conjugates had in vitro cytotoxic activity better than free paclitaxel or free paclitaxel plus free mAb, and also showed high selectivity and specificity towards cells expressing the targeted receptors. In vivo studies showed that paclitaxel•MC192 conjugate had a good antitumor activity while free drugs had no effect at equivalent concentrations.

We also report the chemical coupling of Doxorubicin to an antibody directed to IGF-IR (mAb α-IR3) for the treatment of IGF-IR expressing tumors. The chemical linkage through amine of doxorubicin and aldehydes of the ligand did not affect the binding affinity of mAb αIR3 and allowed to release of the drug inside the target tumor cells. Doxorubicin-mAb conjugate afforded specific and selective toxicity towards cells expressing the targeted receptor. In addition, Doxorubicin-mAb conjugate showed better efficacy in vitro than equimolar concentrations of free drug or free drug plus free mAb. Moreover, the conjugate bypasses p-glycoprotein-mediated resistance to doxorubicin in tumor cells.

In vivo using a model of human tumors xenografted in nude mice, Doxorubicin-mAb conjugate was more efficient at preventing tumor growth and prolonging survival compared to high doses of free doxorubicin or free doxorubicin plus free antibody. Therapy of both the doxorubicin sensitive and resistant tumors were enhanced by the conjugate.

These studies will result in an increase or an improvement of the armamentarium and selectivity of cytotoxic agents. Combinations of other chemotherapeutic agents and other ligands using this approach will generate a several fold increase in the number of anti-tumor agents.

Another embodiment of the present invention can be provided with protecting agents for specific cells.

The target cell surface marker selected corresponds to the receptor for Nerve Growth Factor: the p140 TrkA tyrosine kinase high affinity receptor. TrkA receptors are expressed on normal cells such as neurons (Saragovi, H. U., and Gehring, K. Trends Pharmacol Sci. 21:93-98, 2000). Monoclonal antibodies have been developed against TrkA, namely mAb 5C3, LeSauteur, L. et al. J Neurosci. 16:1308-1316, 1996.

We also report the chemical coupling of a caspase inhibitor peptide (zVAD) to a mAb 5C3 directed to TrkA for the selective protection of apoptotic death in TrkA-expressing neurons. The chemical linkage through thiols exposed on the ligand did not affect the binding affinity of mAb 5C3 and allowed to release of the drug inside the target cells. VAD-mAb conjugate afforded specific and selective protection of caspase-mediated apoptosis towards cells expressing the targeted receptor.

Materials and Methods

Synthesis of 2′Glutaryl-Paclitaxel, and Conjugation to Antibodies

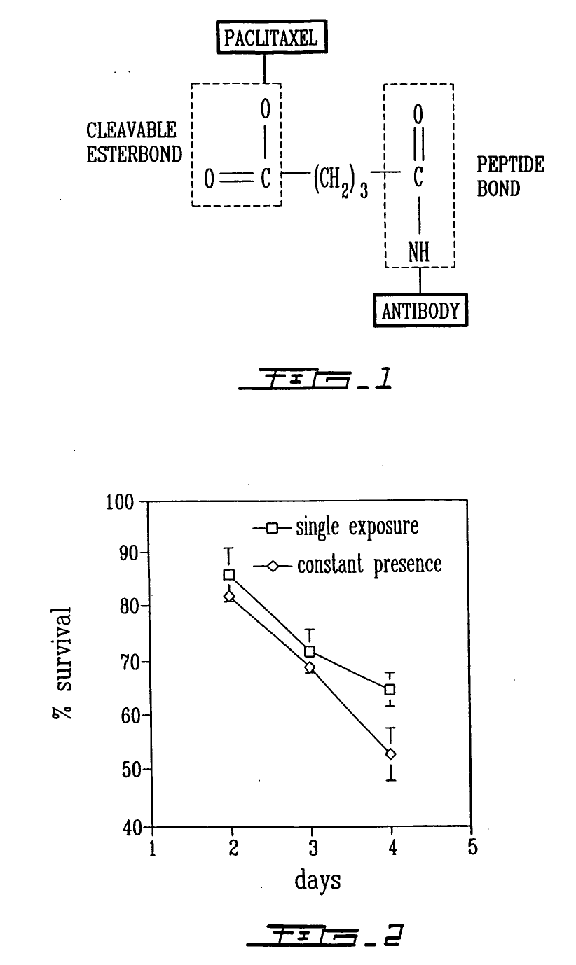

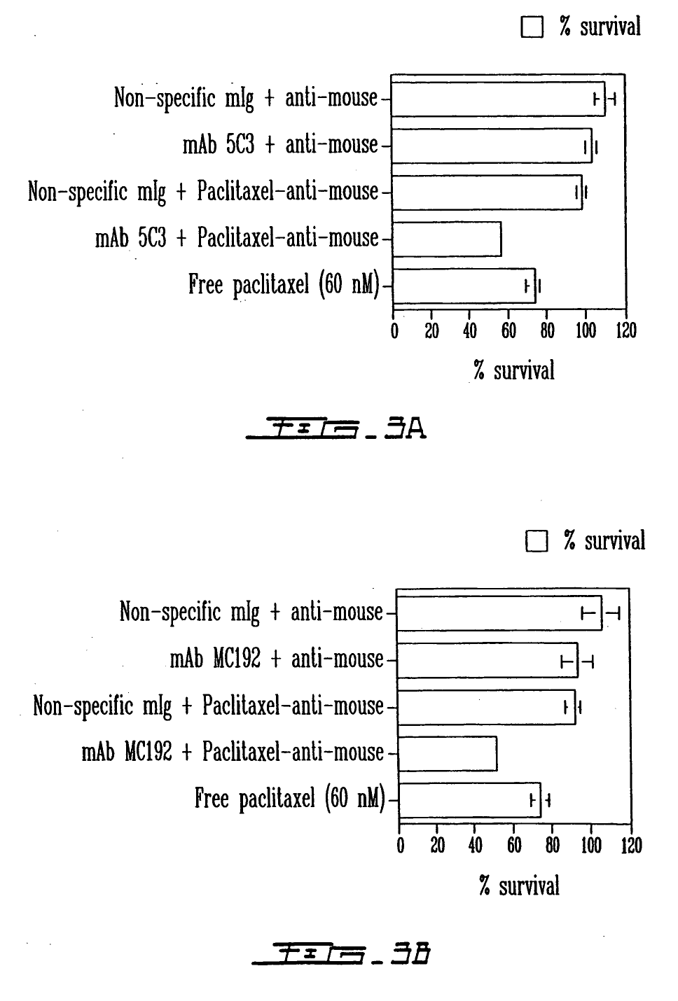

2′glutaryl-paclitaxel was synthetized by mixing 39 μM paclitaxel (Sigma) with 3 μM glutaric anhydride (Sigma), each dissolved in pyridine, for 3 hours at room temperature. This reaction forms an ester bond at the C2′ position of paclitaxel (FIG. 1). The solvent was then removed in vacuo and the residue was dissolved in CHCl₃ and washed with ddH₂O. Purification was achieved by HPLC on a semi-preparative column (Phenomenex); the mobile phase consisted of acetonitrile/water gradient from 35:65 to 75:25 over 50 minutes.

2′glutaryl-paclitaxel (1.334 nmol) was then derivatized with N,N′-carbonyldiimidazole (13.34 nmol) (Sigma) for 25 minutes at 45° C. The carbodiimide reaction activates a carboxylic group on 2′glutaryl-paclitaxel by removing an hydroxyl. Then, antibody was added slowly over a 20 minute period at room temperature at a 2:1 molar ratio of paclitaxel/antibody, and the reaction proceeded for 16 h at 4° C. The reaction forms a paclitaxel-antibody conjugate via formation of a peptide bond with amino groups in the protein (FIG. 1). The solution was then dialyzed for 2 hours against water and overnight against PBS. The conjugate can internalize into cells by receptor-mediated and capping mechanisms.

Synthesis of Doxorubicin-mAb α-IR3

MAb α-IR3 (100 μg) was derivatized with 10 mM sodium periodate in 0.1M acetate buffer pH 5.5 for 30 minutes at room temperature. The oxidation reaction was stopped by adding 15 ml of ethylene glycol for 10 minutes at room temperature. This reaction led to the formation of reactive aldehyde groups.

The by-products were removed by size exclusion and the buffer was exchanged to 1M carbonate/bicarbonate buffer, pH 9.0 using Centricon device (cut off 50, 000 Da).

Doxorubicin (2 μg) was added, and the reaction proceeded for 16 h at 4° C. This reaction formed a Doxorubicin-mAb α-IR3 conjugate via formation of Schiff's bases between reactive aldehydes of the mAb and amino group of the drug. The conjugate was then reduced using 50 mM pyridine borane for 24 h at 4° C. to stabilize the Schiff's bases leading to the formation of a C—N linkage between the mAb and the drug. The conjugate was finally dialyzed for 2 hours against water and overnight against PBS (FIG. 8).

Quantification of Conjugated Paclitaxel

A known mass of paclitaxel-antibody conjugate was incubated for 48 hrs at room temperature in 0.1 M acetate buffer pH 4 to hydrolyze ester bonds. Paclitaxel was then extracted with chloroform and evaporated to dryness. Quantification of thus purified paclitaxel was done by analytical HPLC (Phenomenex), on a mobile phase of acetonitrile/water from 35:65 to 75:25 over 40 minutes. Known concentrations of paclitaxel were used as standard control. Generally, the measured molar ratio of protein to coupled paclitaxel was 1:1, meaning that 1 molecule of paclitaxel coupled to 1 molecule of antibody. Thus, theoretically 1 mole of paclitaxel is delivered per mole of internalized antibody.

Quantification of Conjugated Doxorubicin

Quantification of bound Doxorubicin was done using spectroscopy at 495 nM. Known concentrations of Doxorubicin were used as standard control. The measured molar ratio of mAb to bound Doxorubicin was 1:2, meaning that 2 molecules of Doxorubicin coupled to 1 molecule of mAb.

Cell Lines

The B104 cells are a rat neuroblastoma line that expresses p75 receptors (p75⁺). The 4-3.6 cells are B104 cells stably transfected with human TrkA cDNA (p75⁺, TrkA⁺). NIH 3T3 are mouse fibroblasts that do not express either p75 or TrkA. All cells were cultured in RPMI 1640 supplemented with 5% FBS, L-glutamine, HEPES buffer, and antibiotics.

NIH 3T3 cells are mouse fibroblasts that do not express IGF-IR. The NWT-b3 cells are NIH 3T3 cells stably transfected with human IGF-IR cDNA. KB cells are a human nasopharyngeal cancer cell line that overexpress IGF-IR and KB-V cells are KB cells resistant to drugs after selection with a constant exposure to Doxorubicin. The mechanism of resistance by KB-V is overexpression of pgp (MDR). All cells were cultured in RPMI 1640 supplemented with 5% FBS, L-glutamine, HEPES buffer, and antibiotics.

Antibodies

MAb MC192 is a mouse IgG1 anti-rat p75 mAb Chandler, C. E. et al. J Biol Chem. 259:6882-6889, 1984 and mAb 5C3 is a mouse IgG1 anti-human TrkA mAb LeSauteur, L. et al. J Neurosci. 16:1308-1316, 1996. MC192 and mAb 5C3 were purified and used in culture at 1 nM-5 nM which are near saturating concentrations for cell surface receptors. The “all purpose” secondary rabbit anti-mouse IgG (Sigma) was used in culture at a final concentration of 30 nM.Mab α-IR3 is a mouse anti-human IGF-IR antibody.

Binding Profiles of the Conjugated Antibodies

FACscan assays were used to measure the receptor binding properties of the conjugated antibodies. For testing p75 receptor binding by paclitaxel•MC192, B104 cells in binding buffer (BB: HBSS, 0.1% bovine serum albumine (BSA), 0.1% NaN₃) were incubated with the indicated concentration of either paclitaxel•MC192 (test), intact MC192 (positive control), or mouse IgG (negative control) as primary antibodies; followed by immunostaining with fluoresceinated goat anti-mouse IgG as described. For testing binding of the paclitaxel-rabbit-anti-mouse conjugate, 4-3.6 cells were incubated as above with saturating mAb 5C3 followed by the paclitaxel•rabbit-anti-mouse conjugate (test) or rabbit-anti-mouse antibody (positive control). Cells were then immunostained with fluoresceinated-goat-anti-rabbit IgG.

Binding Profile of the Bound mAb α-IR3

For testing IGF-IR receptor binding by Doxorubicin-mAb α-IR3 conjugate, NWT-b3 cells in binding buffer (BB: HBSS, 0.1% bovine serum albumin (BSA), 0.1% NaN3) were incubated with the indicated concentration of either Doxorubicin- mAb α-IR3 conjugate (test), intact mAb α-IR3 (positive control), or mouse IgG (negative control) as primary antibodies, followed by immunostaining with fluoresceinated goat anti-mouse IgG All data (5,000 cells/point) were acquired on a FACScan and analyzed using the LYSIS II program. Data are reported as mean channel fluorescence (MCF) of bell-shaped histograms.

Kinetics of Paclitaxel Cytotoxicity: Single Bolus Versus Constant Exposure

It is likely that a single bolus of paclitaxel•antibody conjugate would be delivered because affected cells would not synthesize additional target receptor. Therefore we tested whether a single bolus of paclitaxel would be an effective cytotoxic agent. Cells were exposed to the indicated concentration of paclitaxel for 30 minutes at 4° C. Then, cells were plated in a 96-well plate (Falcon); this group represents treatment with paclitaxel present in a constant manner. The remaining cells were washed free of excess paclitaxel prior to plating; this group represents a single exposure to paclitaxel. The survival profile of the cells was measured using the tetrazolium salt reagent 3-(4,5-dimethylthiazol-2-yl)-2,5-diphenyltetrazolium bromide (MTT, Sigma) 48, 72 and 96 hr after plating. Optical density readings of MTT were done in an EIA Plate Reader model 2550 (Bio-Rad) at 595 nm.

In Vitro Cytotoxicity of the Paclitaxel•Antibody Conjugates

For testing the paclitaxel•MC192 conjugate, cells in 96-well plates (2,500-5,000 cells/well) were exposed to either MC192, paclitaxel•MC192 conjugates, or controls. Competition of paclitaxel•MC192 cytotoxicity was done by adding a 4-fold molar excess of MC192 antibody. The survival profile of the cells was measured with the MTT assay 72 hr after plating.

For testing the “all purpose” rabbit-anti-mouse reagent, cells were first exposed to primary mouse mAb 5C3, to mAb MC192, or controls (e.g. mouse IgG). Then, paclitaxel·rabbit-anti-mouse conjugate (test) or rabbit-anti-mouse antibody (negative control) were added to the cultures. The survival profile of the cells was measured with the MTT assay 48 hr after plating.

Mechanism of Action of Paclitaxel•Antibody Conjugates

B104 cells were plated, 25,000 cells/well in a 48 well plate (Falcon). Free paclitaxel (80 nM) or paclitaxel•MC192 (40 nM paclitaxel-equivalent) was added to the well and the cells were incubated for 24 hrs. Cells were then treated with Triton 0.01%, 0.1% sodium citrate and 1 μg DNAse-free RNAse for 1 hour at 0° C. Nuclei were collected following centrifugation. The DNA was labeled with 75 μl propidium iodide (1 mg/ml stock) in 400 μl FACS buffer. All data (3,000 cells/point) were acquired as described above. Paclitaxel release from the conjugate occurs by hydrolysis of the ester bond in the lysosomal compartment Mariani, G. et al. J Nucl Biol Med. 35:111-119, 1991.

In Vitro Cytotoxicity of the Doxorubicin-mAb α-IR3 Conjugate

MTT Assay

For testing the Doxorubicin-mAb α-IR3 conjugate, cells in 96-well plates (1500-2500 cells/well) were exposed to either α-IR3, Doxorubicin-mAb α-IR3 conjugate, or controls in the presence or absence of Verapamil (6 μM). Competition of Doxorubicin-α-IR3 cytotoxicity was done by adding a 10-fold molar excess of α-IR3 antibody. The survival profile of the cells was measured with the MTT assay 72 hr after plating.

Conjugation of mAb 5C3 and a Caspase Inhibitor

Caspase Inhibitor

zVAD=benzoic acid-Valine-Alanine-Aspartic acid-O-methyl-fluoromethylketone peptide. A known caspase inhibitor. It does not penetrate through the plasma membrane.

Conjugation

The ligand (mAb) is derivatized with 10 fold molar excess of SPDP (3-(2-pyridyldithio)propionic acid N-hydroxy-succinimide ester, Pierce) in 5% ethanol/PBS solution (pH 7.4) for 30 min at room temperature. The mixture is then chilled on ice and is reduced with DTT (1,4-dithiothreitol) (5 molar excess DTT with respect to SPDP) for 15 minutes. This reaction reduces SPDP and yields a ligand-SH+PDP. The derivatized ligand is purified by removal of excess DTT and SPDP (Centricon, 50,000 cutoff).

Then the benzoic acid in the zVAD peptide is deprotected with Pd and activated charcoal added in ammonium formate/acetic acid/methanol. The resulting amine in this peptide is then derivatized with SPDP as described above for the ligand. Peptide-S-PDP is purified by HPLC.

Then the 2 agents (ligand-SH and peptide-S-PDP) are mixed in PBS (peptide-S-PDP in molar excess). Oxidation overnight displaces the PDP from the peptide and yields a ligand-caspase inhibitor covalent conjugate via a disulfide bond: ligand-S-S-peptide+PDP byproduct.

In Vitro Survival Assays with 5C3-Caspase Inhibitor

4-3.6 cells are placed in serum free media (SFM, PFHM-II, GIBCO, Toronto), where they undergo apoptosis (serum-withdrawal model).

Cells were then cultured for ˜48 hrs. Cell growth/survival was calculated relative to 5% serum (standardized to 100%). Addition of serum protects cells in SFM from death. Addition of zVAD does not protect cells because it does not penetrate the plasma membrane. The ligand-zVAD conjugate is protective to a much higher degree than the ligand alone. B104 cells that do not express the receptor for the ligand are not protected by the conjugate.

Cell survival was measured by quantitative tetrazolium salt reagent (MTT, Sigma) and optical density (OD) readings as described [Maliartchouk, 1997]. n=3-6.

In Vivo Tumor Studies for MC192-Taxol

Nude mice (seven weeks old, female) were used to test the effect of paclitaxel or conjugates in tumor progression. Single cell suspensions of B104 cells (10⁵/mouse) were injected subcutaneously in the left flank near the rib cage. Tumor growth was monitored daily. After 4 days the tumor volume in all animals approximated 2 mm³. Mice were then randomized and treatments were initiated in four groups (n=5 in each group). Mice in group 1 received saline; mice in group 2 received free paclitaxel (130 ng); mice in group 3 received free paclitaxel (130 ng)+free MC192 (10 μg); mice in group 4 received paclitaxel-MC192 conjugate (65 ng paclitaxel-equivalent and 10 μg MC192-equivalent). All treatments were done by a total of five injections every two days (for a total of ten days). All injections were done IP on the right side to prevent direct contact of the agents to the tumor growing subcutaneously, and to assure that systemic circulation of the drugs was achieved. Measurements of tumor volume were taken using calipers, every day post treatment for a total of 25 days.

The timeline was: day −14 injection of tumors subcutaneously, days −10, −8, −6, −4, −2 injection of drugs or controls, days 0-25 measurement of tumor growth daily.

In Vivo Tumor Studies for α-IR3-Doxorubicin

First, Nude mice (6-8 weeks old, female) were used to establish relative high and low doses of doxorubicin in tumor progression. Then free doxorubicin or conjugates were compared. Single cell suspensions of NWT-b3 (105/mouse), KB or KB-V (2.5×105/mouse) were injected subcutaneously in the left flank near the rib cage. Tumor growth was monitored daily. After the volume of the tumors had reached 2 mm3 , mice were randomized and treatments were initiated in four or five groups (n=5 in each group). Mice received either saline, low dose of doxorubicin (DL; 0.32 micrograms per injection), high dose of doxorubicin (DH; 160 micrograms per injection), non-conjugated DL+αIR3 antibody, or Doxorubicin-αIR3 conjugate (DL-equimolar). All treatments were done by a total of five injections every 2 days (for a total of 10 days). All injections were done IP on the right side to prevent direct contact of the agents to the tumor growing subcutaneously, and to assure that systemic circulation of the drugs had to be achieved. Measurements of tumor volume were taken every 2 days post treatment for a total of 20 days.

Statistical Analysis

Statistical significance of differences in tumor growth among the different treatment groups was determined by the student t test using SYSTAT 7.0 software. P value is significant when it is <0.05.

Results

Kinetics of Paclitaxel Cytotoxicity: Single Bolus Versus Constant Presence

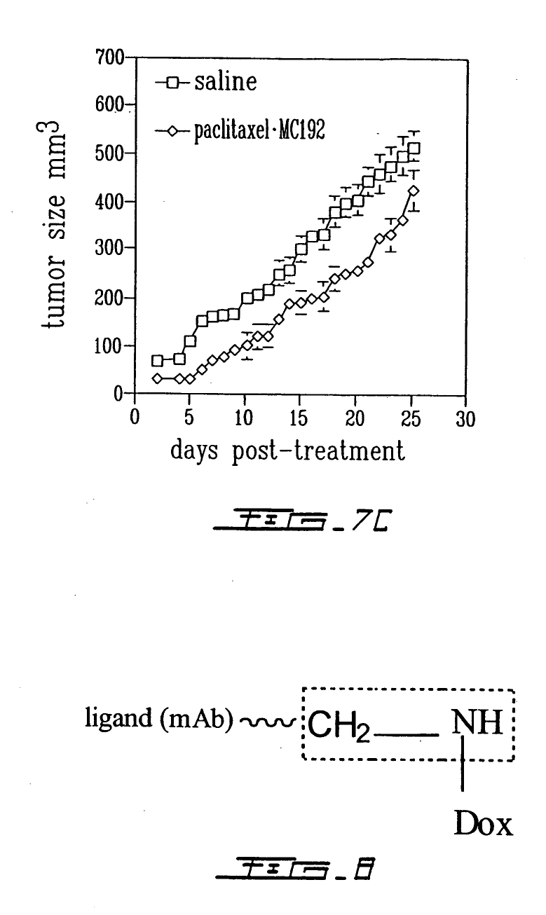

Free paclitaxel is lipophilic and readily penetrates the cell membrane. In contrast, paclitaxel•antibody conjugates penetrate the cell via receptor-mediated internalization. Since it is likely that paclitaxel•antibody conjugate would be delivered as a single bolus (because cells affected would cease synthesis of receptor targets), we tested whether a single short term exposure to paclitaxel can be effective in killing neuroblastoma B104 cells (FIG. 9). These assays were done at 4° C. to allow internalization of drug comparable to that afforded by antibody-mediated delivery of paclitaxel.

The cytotoxic effect of free paclitaxel is generally the same whether the drug is present in the culture throughout or after a single exposure. Comparable killing was verified at several paclitaxel concentrations. However, a single exposure to 20 nM paclitaxel is significantly less effective than constant exposure after 72 and 96 hours of culture. Likely the amount of drug taken up by the cells after 30 min exposure to 20 nM paclitaxel is sufficient to kill cells over a period of 2 days but not for longer times. These results were encouraging because the cytotoxicity of paclitaxel-antibody conjugates would be similar to that seen after short-term or single exposure to free paclitaxel. Similar data were obtained with 4-3.6 cells.

Binding and Cytotoxicity of Paclitaxel-Conjugated Antibodies

To assess whether paclitaxel conjugation to antibodies affected antibody binding, this property was tested in FACScan assays (Table 1). Conjugated paclitaxel•rabbit anti-mouse lost only ˜20% of the binding activity compared to unconjugated rabbit-anti-mouse antibody. The binding activity of conjugated paclitaxel•MC192 was intact, compared to unconjugated MC192. These results indicate that the method used to conjugate paclitaxel to antibody in a 1:1 ratio does not affect significantly the binding properties of the antibodies.

TABLE I
Efficient target binding by paclitaxel•antibody conjugates

Binding Fluorescence

| Experimental Conditions | 4-3.6 cells | B104 cells |
|---|---|---|
| Background staining | 4 ± 3 | 3 ± 0 |
| Paclitaxel-coupled antibody | 81 ± 28 | 130 ± 7 |
| Intact antibody | 100 | 100 |
| 4-3.6 cells were bound with mAb 5C3 (10 μg/ml), followed by paclitaxel•rabbit-anti-mouse or intact rabbit-anti-mouse, and goat-anti-rabbit-FITC. B104 cells were bound with paclitaxel•MC192 (10 μg/ml), or unconjugated MC192 (10 μg/ml), followed by goat-anti-mouse-FITC. Background was assessed by replacing the primary mAb with mouse IgG (10 μg/ml). Cells were analyzed (5,000/assay) by FACScan and LYSIS II software. The | | |
| #data are mean channel fluorescence of bell-shaped histograms, stan staining by unconjugated primary antibody ± sem. n = 4. | | |

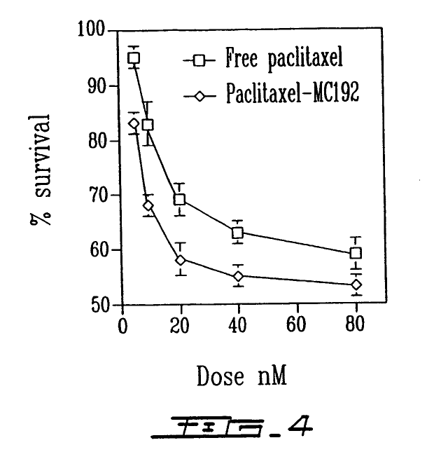

The cytotoxic activity of the “all purpose” paclitaxel-anti-mouse conjugate was evaluated in vitro against neuroblastoma cells (FIG. 3). The paclitaxel•rabbit-anti-mouse conjugate was active against cells only when a specific mouse primary antibody was present: mAb 5C3 that binds 4-3.6 cells (FIG. 3A) and mAb MC192 that binds B104 cells (FIG. 3B). Cytotoxicity was better and more selective than equimolar doses of free paclitaxel. In control assays conjugate paclitaxel•rabbit-anti-mouse in the presence of a non-specific primary was not cytotoxic, and the specific primary mAbs in the presence of unconjugated rabbit-anti-mouse were not cytotoxic. Similar analysis using paclitaxel•MC192 conjugates also showed better activity and selectivity than free paclitaxel at equimolar concentrations (FIG. 4).

These data suggest that the paclitaxel•rabbit anti-mouse conjugate was active by binding to the specific primary antibody, while paclitaxel•MC192 conjugates were active by directly targeting p75 receptors. Presumably the conjugates internalized and released the cytotoxic agent inside the cells. Because only a fraction of paclitaxel•antibody conjugates can internalize via the targeted receptor, the data suggest that conjugates may be significantly much better at cell killing than free paclitaxel, possibly because of improved transport or penetration. Paclitaxel release after hydrolysis of the conjugate could not be measured directly in vivo because of technical limitations.

Binding and Cytotoxicity of Doxorubicin-Conjugated Antibodies

FACScan was used to access whether the α-IR3 antibody was affected by the chemistry used for conjugation (table 2). The conjugated antibody retained its full binding activity after conjugation compared to free α-IR3. This result indicates that, as expected, coupling antibodies through their carbohydrates moieties which are distant from the antigen binding site avoid loss of antibody binding activity.

TABLE 2
Efficient target binding by Doxorubicin-mAb conjugate

| Experimental conditions | Binding fluorescence |
|---|---|
| Background staining | 10 ± 1 |
| Doxorubicin-mAb | 101 ± 2 |
| Intact mAb | 100 |

NWT-b3 cells were bound with unconjugated mAb αIR3 (10 μg/ml), or Doxorubicin-mAb (10 μg/ml mAb equivalent) conjugate, followed by goat-anti-mouse-FITC. Bavkground was assessed by replacing the primary mAb with mouse IgG (10 μg/ml). Cells were analyzed (5,000/assay) by FACScan and LYSIS II software. The data are mean channel fluorescenc of bell-shaped histograms, standardized to maximal staining by unconjugated primary antibody ± sem. n = 5.

The cytotoxicity of the conjugate was initially evaluated in vitro using mouse fibroblasts stably transfected with-IGF-IR (FIG. 9). KB and KB-V cells were then used to access cytotoxicity as well and to check whether the conjugate could bypass p-glycoprotein-mediated resistance (table 3). Cytotoxicity was better than equimolar doses of free Doxorubicin as well as free Doxorubicin plus free antibody while the antibody alone showed no effect on those cells. The conjugate showed unaltered cytotoxicity on KB-V cells which are multidrug resistant as compared to the sensitive KB cells with or without the channel inhibitor Verapamil while free Doxorubicin was inactive on KB-V cells unless Verapamil was added. The results are consistent whether MTT or Colony Formation Assay is done to measure cell death.

TABLE 3
Bypassing of the p-glycoprotein-mediated resistance by the
conjugate

IC50 (nm) ± SEM

| Treatment | KB cells | KB-V cells |
|---|---|---|
| Doxorubicin | 60 ± 2 | >320 |
| Dox + mAb | 60 ± 2 | >320 |
| Dox + Verapamil | 60 ± 1 | 150 ± 1 |
| Dox − mAb | 25 ± 3 | 30 ± 4 |
| Dox-mAb + Verapamil | 31 ± 1 | 30 ± 2 |

KB and KB-V cells were treated for 3 days with different concentrations of Doxorubicin, Doxorubicin plus mAb or Doxorubicin-mAb conjugate with or without 6 μM of the p-glycoprotein inhibitor Verapamil. Percent metabolic activity ± sem was determined by standardizing untreated cells to 100%. n = 3. Representative of 4 independent experiments.

Those results indicate that the conjugate can bypass p-glycoprotein-mediated resistance by delivering doxorubicin into the cell by a mechanism not associated with diffussion.

Selectivity and Specificity of Paclitaxel•MC192 Conjugates

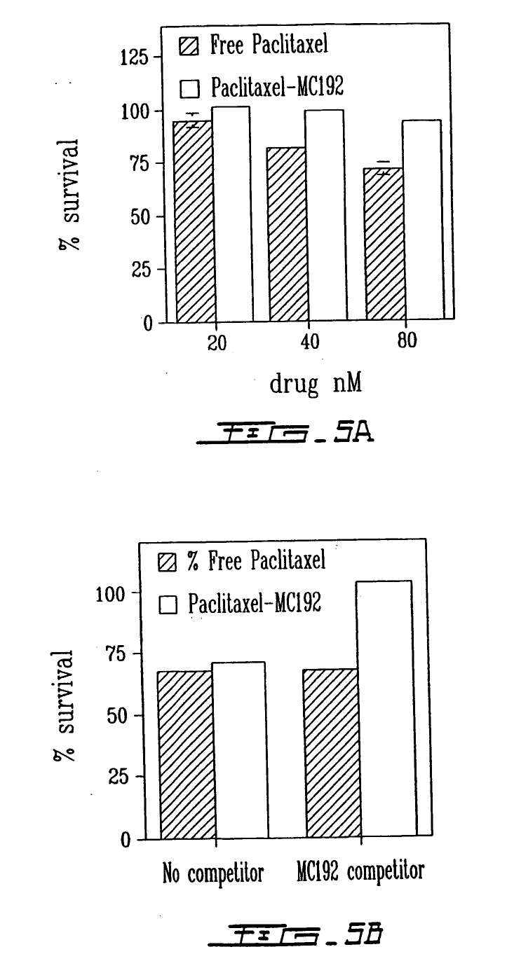

The selectivity of paclitaxel•MC192 was evaluated using cells that do not express p75 (FIG. 5A). The results show that the conjugate was inactive, while free paclitaxel exhibited dose-dependent cytotoxicity. These results suggest that the activity of paclitaxel•MC192 conjugates is selective towards cells expressing p75 receptors. The specificity of paclitaxel•MC192 conjugate was investigated by ligand competition (FIG. 5B). At 10 nM paclitaxel•MC192 conjugate (10 nM paclitaxel-equivalent) there is efficient killing of B104 cells. Concomitant addition of 40 nM MC192 blocks cytotoxicity by competing for the p75 receptor target. In contrast, addition of 40 nM non-specific mouse IgG does not affect the activity of paclitaxel•MC192 conjugates. Cold competition of paclitaxel•MC192 indicates that death is mediated specifically via p75 receptors. Furthermore, free paclitaxel had the same cytotoxicity whether or not 40 nM of mouse IgG or 40 nM of MC192 antibody were added to the cultures.

Because some antibodies increase free drug-mediated killing compared to drugs alone, we investigated whether MC192 had a pharmacological role as adjuvant. MC192 mAb or mouse IgG did not enhance or decrease the cytotoxicity of various concentrations of free paclitaxel. Similar data were obtained with 60 nM free paclitaxel cultured with increasing doses of antibody. These results indicate that MC192 did not have a pharmacological role, and suggest that MC192 acts only as a carrier and does not contribute to the cytotoxicity of the conjugate in vitro.

Selectivity and Specificity of Doxorubicin-αIR3

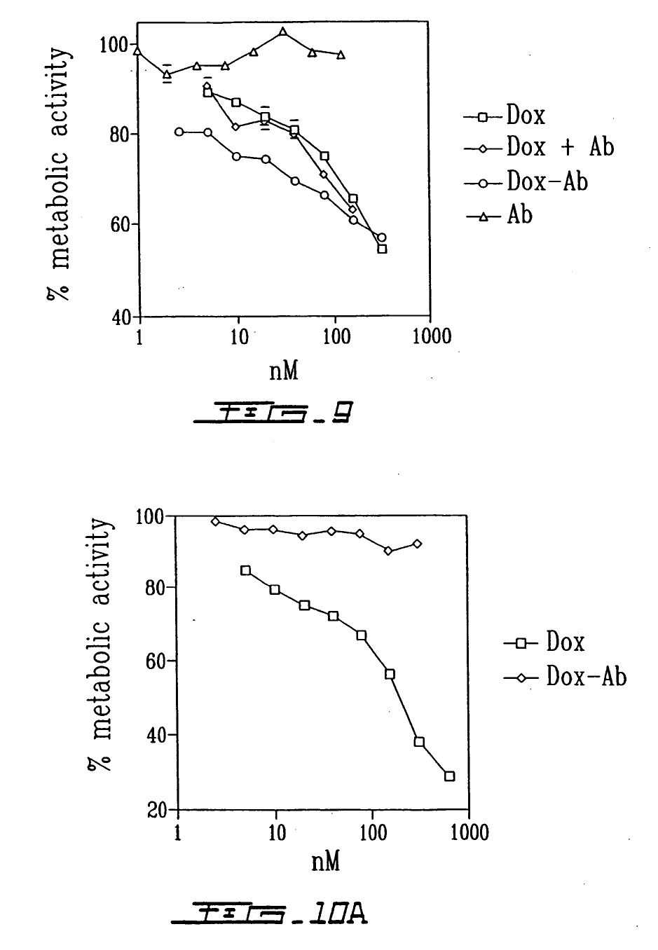

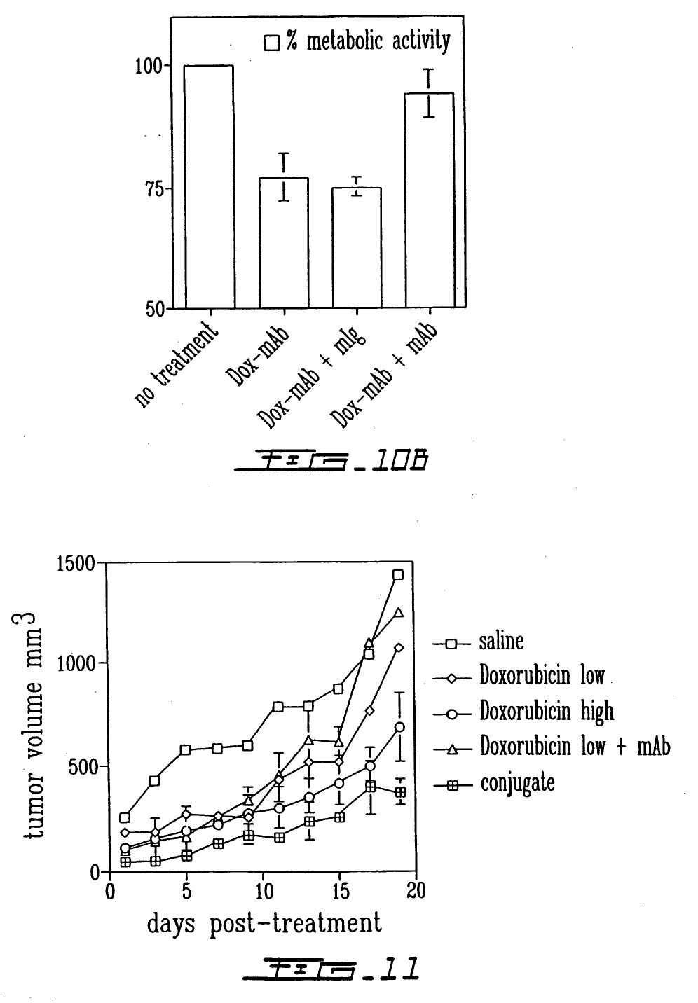

The selectivity of the conjugate was evaluated using mouse fibroblasts which do not express IGF-IR receptors (FIG. 10A). The conjugate was totally inactive on those cells, while free Doxorubicin exhibited a dose-dependent cytotoxicity. This result indicates that the conjugate is selective towards cells expressing IGF-IR, the targeted receptor. The specificity of the conjugate was evaluated by ligand competition using mouse fibroblasts stably transfected with IGF-IR (FIG. 10B). There was efficient killing by the conjugate which was abolished in the presence of 10 molar excess free α-IR3. Addition of an excess of mlg did not change the efficiency of the conjugate. Competition of Doxorubicin-αIR3 indicates that the killing is specifically mediated by IGF-IR receptors. Free Doxorubicin had the same cytotoxicity whether excess of mlg or α-IR3 was present or not.

Cytotoxic Mechanism of Paclitaxel•MC192 Conjugates

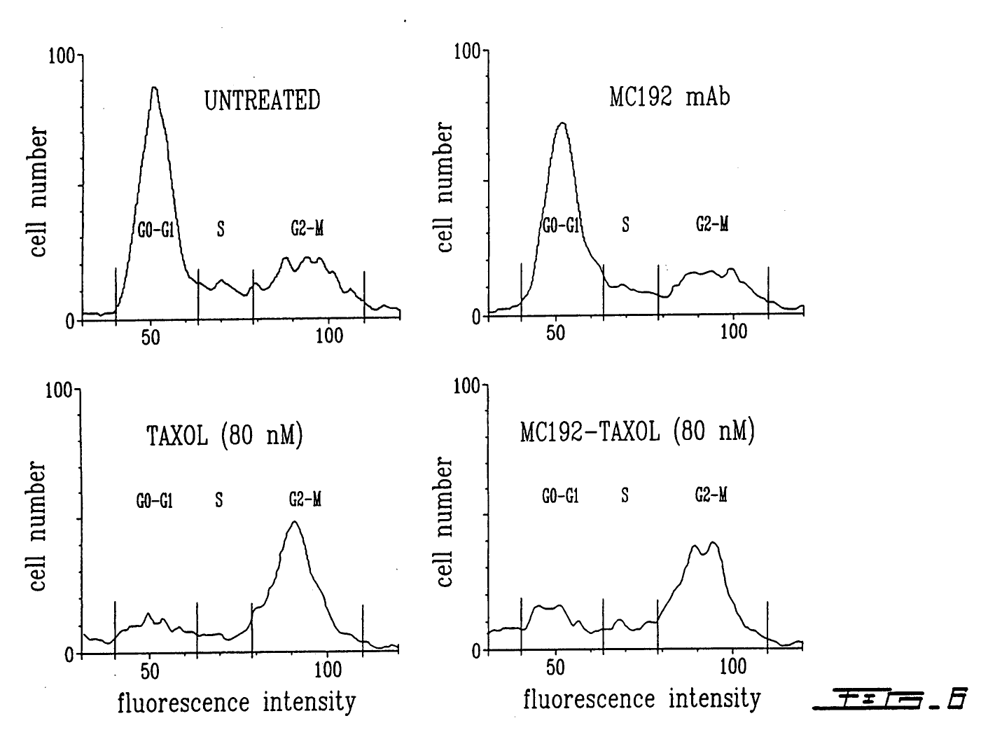

To assess whether the mechanism of action of paclitaxel•MC192 conjugates is the same as free paclitaxel, cell cycle analysis was done in FACScan assays (FIG. 6). The data show that paclitaxel•MC192 conjugates arrest cells in the G2-M phase of the cell cycle which is consistent with the mechanism of action of free paclitaxel. A G2-M arrest leads to apoptosis in these cells. MC192-treated cells cycle is like untreated control, indicating no effect by the antibody.

In Vivo Activity of Paclitaxel•MC192

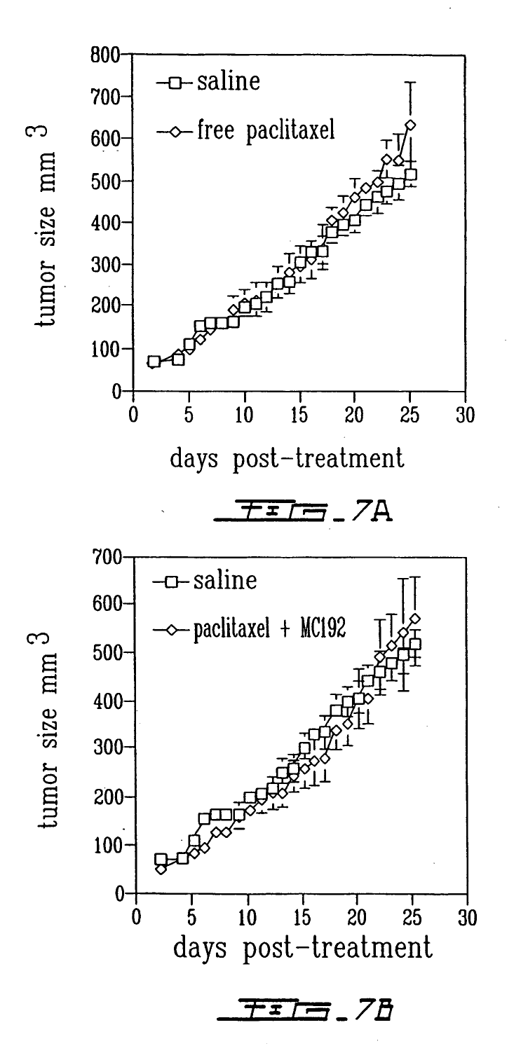

The antitumor activity of paclitaxel•MC192 was evaluated in vivo against neuroblastoma xenografted in nude mice (FIG. 7). The results show that the conjugate was effective in reducing tumor growth compared to the control (HBSS) (t test, P<0.05) (FIG. 7C), while paclitaxel alone or in combination with MC192 were not able to do so (FIGS. 7A, 7B). Moreover, the conjugate prolonged the survival of the mice on average by ˜30% compared to free paclitaxel.

In Vivo Efficacy of the Doxorubicin-αIR3 Conjugate

The antitumor efficacy of the conjugate was evaluated using a mouse fibroblast cell line stably transfected with human IGF-IR (NWT-b3) (FIG. 11) and also in KB cells either sensitive or resistant (KB-V) to doxorubicin, xenografted in nude mice. The results showed that the conjugate was more effective at reducing tumor growth, compared versus free doxorubicin dosed at 50× higher molar concentration. Survival was also enhanced.

Selective Protection by Selective Inhibition of Caspase

The efficacy of a mAb 5C3-VAD conjugate was tested versus 4-3.6 cells that express the target TrkA (bound by mAb 5C3) and versus B104 cells that do not express TrkA. Results are shown in Table 4.

TABLE 4

| | 4-3.6 Cell Survival |
|---|---|
| | (MTT, % of serum |
| | control) |
| Serum Free media | 0 ± 4 |
| 5% serum | 100 ± 2 |
| 20 nM zVAD | −2 ± 3 |
| VAD-5C3 conjugate (20 nM VAD equivalent, 1 nM | 41 ± 1 |
| 5C3) | |

Neither 20 nM zVAD nor VAD-5c3 conjugate afford protection to B104 cells (parental cells to 4-3.6, but TrkA). MAb 5C3 does not bind to protect B104 cells, and does not protect these cells from death. Typicaly, 0.1 nM mAb 5C3 alone (unconjugated) does protect 4-3.6 cells in SFM to ˜10%, which is a much lower degree of protection than equimolar doses of the VAD-5C3 conjugate.

Discussion

We have shown that mAb MC192 and mAb 5C3, ligands for p75 and trka receptors respectively, can be used as carriers for paclitaxel to afford efficient and specific tumor toxicity. We also show that an “all purpose” targeting agent can be developed by paclitaxel conjugation of anti-Ig secondary antibodies.

Kinetics of paclitaxel cytotoxicity. Since the cytotoxicity of paclitaxel•antibody conjugates was expected to be similar to that seen after short-term or single exposure to free paclitaxel, we first assessed whether a single dose of paclitaxel could be efficient at killing the cells. We demonstrated that paclitaxel cytotoxicity is the same after 48 hours whether a constant or a single dose is given in vitro. This finding underlines the clinical experience of paclitaxel delivery, which is often delivered as a single bolus every few weeks, unlike many other chemotherapeutics which are most effective when delivered at low doses over prolonged periods.

Criteria for conjugation. We set out to fulfill several criteria that would improve the therapeutic index of paclitaxel: (a) to chemically conjugate mAbs to taxanes; (b) to afford conjugates that are highly soluble in physiological buffers; (c) chemical coupling should not affect mAb targeting function, but should result in a prodrug; (d) after binding to the target receptor mAbs should induce internalization and release the drug. All of these criteria were fulfilled.

Paclitaxel was linked via its most reactive hydroxyl group (C2′ position) to antibodies as carriers. This esterification leads to paclitaxel inactivation because this position is crucial for tubulin binding, thus the conjugate is a prodrug. The binding activity of the antibodies were essentially preserved after coupling; only ˜20% loss of binding was observed for the “all purpose reagent” and no loss for MC192. After hydrolysis of the conjugate, active paclitaxel is released in sufficient amounts to kill cells. As expected, the conjugates arrest the cells in prophase, like paclitaxel does.

Improved efficacy. The cytotoxic activity of the conjugates was better than that of free paclitaxel. This may be due to better transport, penetration and accumulation of the drug inside the cells. Moreover, conjugate cytotoxicity related well with the density of target receptors on the cell lines. Although it has been suggested that in certain systems ligand-bound p75 receptors may activate ceramide pathways and pro-apoptotic signals, the carrier itself had no direct pharmacological role. Hence cytotoxicity is only due to paclitaxel.

Improved selectivity. No binding of the conjugates were observed in cells that do not express the target receptors. We selected the paclitaxel•MC192 conjugate for in vivo experiments because it was more suitable than the “all purpose” paclitaxel•rabbit anti-mouse conjugate. The in vivo experiments confirmed our in vitro findings. The paclitaxel•MC192 conjugate had a significant antitumor activity against cells expressing p75 receptor in the experimental model used and we observed a delay in tumor growth compared to other groups.

The efficacy of the conjugate compared to free drug was much more evident in vivo than in vitro, probably because in vivo the conjugate was concentrated at the tumor site. The effective concentration of conjugate tested in vivo was ˜3.5 nM, whereas free paclitaxel was not effective at this dose. Since the effective concentration of taxanes in humans is in the millimolar range, the therapeutic index of the conjugate is improved several fold. Furthermore, we demonstrated appropriate in vivo systemic distribution and pharmacokinetics for the conjugate.

We did not observe any obvious toxicity in the treated animals, and we believe that the paclitaxel•MC192 conjugate is much less toxic than free paclitaxel as it spares non-target expressing cells.

All the reasons mentioned above make the paclitaxel•MC192 conjugate a potential candidate for the treatment of p75-expressing tumors. The conjugates are not only better cytotoxic agents than free paclitaxel but they are also highly water-soluble which is a great advantage considering the severe hypersensitivity reactions experienced by paclitaxel-treated patients. The properties of the conjugates may make them interesting therapeutic agents to add to the chemotherapeutic armamentarium.

Paclitaxel is frequently given in combination with antibodies but not physically bound to them; here, we report the first synthesis of paclitaxel•antibody conjugates. A general method is proposed to selectively target cancer cells by concentrating cytotoxic drugs at the tumor site, and inside the tumors. Furthermore, it would be interesting to couple cytotoxic drugs to small peptidic or non-peptidic ligands of tumor markers to overcome obstacles (such as proteolysis, immunogenicity, and poor penetration of solid tumors) inherited to antibodies and proteins Saragovi, H. U., and Gehring, K. Trends Pharmacol Sci. 21:93-98, 2000 when used as therapeutics.

In vitro, the ligand-doxorubicin conjugate also demonstrated similar efficacy as free doxorubicin. However the conjugate bypassed pgp resistance to drug. In vivo the conjugate was significantly more effective than 50× higher molar concentration of free drug.

In vitro, the ligand-caspase inhibitor conjugate demonstrated higher efficacy than free peptide caspase inhibitor because the free peptide can not enter the cell and target the caspases which are located inside the cell. Selective protection of apoptosis was thereby achieved.

We propose that reduced toxicity and improved therapeutics can be achieved by selective delivery of toxic anti-cancer agents to tumor cells, this is achieved because the effective dose of chemotherapy may be reduce, because non-tumor cells are largely spared, and because resistance to chemotherapy may be expanded.

We also proposed that reduced toxicity and improved therapeutics can be achieved by selective delivery of protective agents (such as caspase inhibitors, estrogen analogs, or vitamin D analogs) to non-tumor cells such that these cells are spared or protected from death. A person skilled in the art will know that the concept of selective protection can be expanded to degenerative disorders such as Alzheimers disease.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A compound to selectively kill a target cell in a patient with reduced systemic toxicity, which comprises a compound of the formula:
W-Z-X
wherein,
X is a therapeutical agent selected from the group consisting of chemotherapeutic agent, antiviral agent, antibacterial agent, antifungal agent and enzyme inhibitor agent;
W is a molecule which is adapted to selectively bind said target cell directly or indirectly; and
Z is a breakable linker which covalently links W and X together, wherein said linked W remains available for binding to said target cell, whereby said breakable linker releases said therapeutical agent into said target cell.

2. The compound of claim 1, wherein said compound when bound to said target cell is internalized into said target cell.

3. The compound of any one of claims 1 and 2, wherein said linker is breakable by pH modification, reduction or enzymatic hydrolysis.

4. The compound of any one of claims 1 to 3, wherein said chemotherapeutic agent is selected from the group of taxanes, taxanes derivatives, anthracyclines, anthracyclines derivatives, doxorubicin, daunomycin, daunorubicin, adriamycin, methotrexate, mitomycin, epirubicin, nucleoside analogs, DNA damaging agents and tyrphostins.

5. The compound of any one of claims 1 to 4, wherein said therapeutical agent is selected from the group of antisense oligonucleotide and cDNA for a gene.

6. The compound of claim 4 wherein said taxane is paclitaxel.

7. The compound of any one of claims 1 to 3, wherein said chemotherapeutic agent is doxorubicin.

8. The compound of claim 1 wherein said molecule is selected from the group of antibody and mimicking molecules thereof, peptides, peptidomimetics, growth factors, hormones, adhesion molecules, viral proteins and functional fragments thereof.

9. The compound of claim 8 wherein said antibody is a monoclonal antibody.

10. The compound of claim 8, wherein said antibody binds to a specific receptor on said target cell.

11. The compound of claim 9 wherein said monoclonal antibody is selected from the group of MC192, 5C3 and a-IR3.

12. The compound of claim 1, wherein said compound further comprises a spacer between W and Z and/or between Z and X.

13. The compound of claim 12, wherein when W is a primary biologically active molecule indirectly binding to said target cell, said compound further comprises W′ which is a secondary biologically active molecule selectively bound to W and adapted to selectively bind said target cell.

14. The compound of claim 13 wherein said primary and/or said secondary biologically active molecules is an antibody.

15. The compound of claim 14 wherein a primary antibody is of a species and a secondary antibody is of a different species.

16. The compound of any one of claims 14 or 15, wherein said antibody is a monoclonal antibody.

17. The compound of claim 13 wherein said secondary biologically active molecule is a rabbit-antimouse antibody.

18. The compound of claim 1, wherein said compound is of the formula: 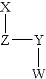
wherein Y is a spacer selected from the group of alkene, alkyl, methyl, ethyl ester, ethyl glycol and H(CH₂CH₂O)ₙOH, n being between 1 and 90.

19. The compound of claim 18, wherein said spacer is (CH₂)₃.

20. The compound of claim 1, wherein said compound is of the formula I,

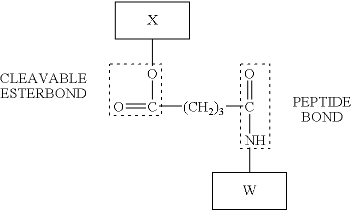

21. The compound of claim 1, wherein Z is

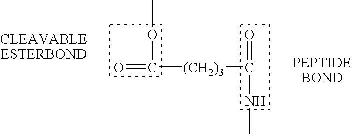

22. The compound of claim 1, wherein said compound is of the formula II,

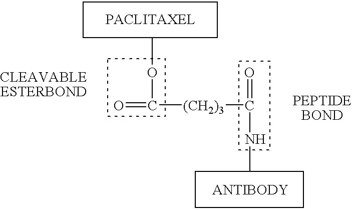

23. The compound of claim 1, wherein said compound is of the formula III,

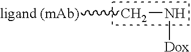

24. A therapeutical composition, which comprises a therapeutically effective amount of a compound of any of claims 1 to 23 in association with a pharmaceutically acceptable carrier.

25. An anti-cancer composition, which comprises a therapeutically effective amount of a compound of any of claims 1 to 23 in association with a pharmaceutically acceptable carrier, wherein said therapeutical agent is a chemotherapeutic agent.

26. A method for treating cancer with reduced effects in a patient, said method consisting in administering a therapeutically effective amount of a compound of any of claims 1 to 23 to a patient, wherein said therapeutical agent is a chemotherapeutic agent.

27. Use of the compound of any one of claims 1 to 23 for the manufacture of a medicament for the treatment of cancer with reduced effects in a patient, wherein said therapeutical agent is a chemotherapeutic agent.

28. A method for decreasing toxic side effects and increasing selectivity of a chemotherapeutic agent for tumor cells, said method comprising the step of administering to a patient a conjugate comprising a chemotherapeutic agent conjugated to a molecule which is adapted to selectively bind said target cell directly or indirectly, wherein said compound when bound to said target cell is internalized into said cell and to a breakable linker which covalently links said molecule and said chemotherapeutic agent together, wherein said linked molecule remains available for binding said target cell, whereby said breakable linker releases said chemotherapeutic agent into said target cell.

29. Use of a chemotherapeutic agent conjugated to a molecule for decreasing toxic side effects and increasing selectivity of a chemotherapeutic agent for tumor cells, said molecule being adapted to selectively bind said target cell directly or indirectly, wherein said compound when bound to said target cell is internalized into said cell and to a breakable linker which covalently links said molecule and said chemotherapeutic agent together, wherein said linked molecule remains available for binding said target cell, whereby said breakable linker releases said chemotherapeutic agent into said target cell.

30. A method for by-passing resistance of tumor cells by p-glycoprotein pump (PGP), said method comprising the step of administering the compound of claim 1 to a patient in need of such a treatment whereby said biologically active molecule is a monoclonal antibody and said compound is avoiding membrane diffusion and/or permeability route to enter into said cells.

31. A compound to selectively protect a target cell which comprises a compound of the formula:
W-Z-X
wherein,
X is a protective agent to cells selected form the group consisting of: enzyme inhibitors, ligands of nuclear receptors, vitamin D, vitamin E and analogs thereof, estrogen and analogs thereof and inhibitors of the apoptotic cascase;
W is a biologically active molecule which is adapted to selectively bind said target cell directly or indirectly; and
Z is a linker which covalently links W and X together, wherein said linked W remains available for binding said target cell, whereby said linker releases said therapeutical agent into said cell and whereby said compound is providing a patient with a reduced systemic toxicity.

32. The compound of claim 31, wherein said protective agent is an enzyme inhibitor agent.

33. The compound of claim 32, wherein said enzyme inhibitor agent is a caspase inhibitor agent.

34. A method for decreasing toxic side effects to non-tumor cells, said method comprising the step of administering to a patient a conjugate comprising a protective agent conjugated to a molecule which is adapted to selectively bind said non-tumor target cell directly or indirectly, wherein said compound when bound to said non-tumor target cell and to a breakable linker which covalently links said molecule and said protective agent together, wherein said linked molecule remains available for binding said target cell, whereby said breakable linker releases said protective agent into said cell and whereby said protective agent internalized in said cell is protecting said cell from subsequent toxicity by a chemotherapeutic agent which is therefore decreasing toxic side effects.
